(19) United States
(12) Patent Application Publication
(10) Pub. No.: US 2023/0390055 A1
(43) Pub. Date: Dec. 7, 2023

(54) Balloon Expandable Leaflet Protection During Crimping

(71) Applicant: St. Jude Medical, Cardiology Division, Inc., St. Paul, MN (US)

(72) Inventors: Tracee Eidenschink, Wayzata, MN (US); Michael Shane Morrissey, St. Paul, MN (US); Jeffrey Paul LaPlante, Minneapolis, MN (US)

(73) Assignee: St. Jude Medical, Cardiology Division, Inc., St. Paul, MN (US)

(21) Appl. No.: 18/327,144

(22) Filed: Jun. 1, 2023

Related U.S. Application Data
(60) Provisional application No. 63/349,241, filed on Jun. 6, 2022.

(51) Int. Cl. A61F 2/24

(52) U.S. Cl. CPC A61F 2/2418; A61F 2/2433; A61F 2220/0075; A61F 2230/0002; A61F 2250/0069

(57) ABSTRACT

According to one aspect of the disclosure, a prosthetic heart valve system includes a prosthetic heart valve including a stent, a cuff and a plurality of leaflets, the cuff and the plurality of leaflets forming a valve assembly, and an expandable balloon having a deflated state and an inflated state, the expandable balloon being configured and arranged to transition the prosthetic heart valve from a collapsed condition to an expanded condition and protect portions of the valve assembly during crimping and delivery.

### CROSS-REFERENCE TO RELATED APPLICATIONS

This application claims priority to U.S. Provisional Patent Application No. 63/349,241, filed Jun. 6, 2022, the disclosure of which is hereby incorporated by reference herein.

### BACKGROUND OF THE DISCLOSURE

Valvular heart disease, and specifically aortic and mitral valve disease, is a significant health issue in the United States. Valve replacement is one option for treating heart valve diseases. Prosthetic heart valves, including surgical heart valves and collapsible/expandable heart valves intended for transcatheter aortic valve replacement (“TAVR”) or transcatheter mitral valve replacement (“TMVR”), are well known in the patent literature. Surgical or mechanical heart valves may be sutured into a native annulus of a patient during an open-heart surgical procedure, for example. Collapsible/expandable heart valves may be delivered into a patient via a tube-like delivery apparatus such as a catheter, a trocar, a laparoscopic instrument, or the like to avoid a more invasive procedure such as full open-chest, open-heart surgery. As used herein, reference to a “collapsible/expandable” heart valve includes heart valves that are formed with a small cross-section that enables them to be delivered into a patient through a tube-like delivery apparatus in a minimally invasive procedure, and then expanded to an operable state once in place, as well as heart valves that, after construction, are first collapsed to a small cross-section for delivery into a patient and then expanded to an operable size once in place in the valve annulus.

Collapsible/expandable prosthetic heart valves typically take the form of a one-way valve structure (often referred to herein as a valve assembly) mounted to/within an expandable stent. In general, these collapsible/expandable heart valves include a self-expanding or balloon-expandable stent, often made of nitinol or another shape-memory metal or metal alloy (for self-expanding stents) or steel or cobalt chromium (for balloon-expandable stents). Existing collapsible/expandable TAVR devices have been known to use different configurations of stent layouts—including straight vertical struts connected by “V”s as illustrated in U.S. Pat. No. 8,454,685, or diamond-shaped cell layouts as illustrated in U.S. Pat. No. 9,326,856, both of which are hereby incorporated herein by reference. The one-way valve assembly mounted to/within the stent includes one or more leaflets, and may also include a cuff or skirt. The cuff may be disposed on the stent's interior or luminal surface, its exterior or abluminal surface, and/or on both surfaces. A cuff helps to ensure that blood does not just flow around the valve leaflets if the valve or valve assembly are not optimally seated in a valve annulus. A cuff, or a portion of a cuff disposed on the exterior of the stent, can help retard leakage around the outside of the valve (the latter known as paravalvular or “PV” leakage).

Balloon expandable valves are typically delivered to the native annulus while collapsed (or “crimped”) onto a deflated balloon of a balloon catheter, with the collapsed valve being either covered or uncovered by an overlying sheath. Once the crimped prosthetic heart valve is positioned within the annulus of the native heart valve that is being replaced, the balloon is inflated to force the balloon expandable valve to transition from the collapsed or crimped condition into an expanded or deployed condition, with the prosthetic heart valve tending to remain in the shape into which it is expanded by the balloon. Typically, when the position of the collapsed prosthetic heart valve is determined to be in the desired position relative to the native annulus (e.g. via visualization under fluoroscopy), a fluid (typically a liquid although gas could be used as well) such as saline is pushed via a syringe (manually, automatically, or semi-automatically) through the balloon catheter to cause the balloon to begin to fill and expand, and thus cause the overlying prosthetic heart valve to expand into the native annulus.

When self-expandable prosthetic heart valves are delivered into a patient to replace a malfunctioning native heart valve, the self-expandable prosthetic heart valve is almost always maintained in the collapsed condition within a capsule of the delivery device. While the capsule may ensure that the prosthetic heart valve does not self-expand prematurely, the overlying capsule (with or without the help of additional internal retaining features) helps ensure that the prosthetic heart valve does not come into contact with any tissue prematurely, as well as helping to make sure that the prosthetic heart valve stays in the desired position and orientation relative to the delivery device during delivery. However, balloon expandable prosthetic heart valves are typically crimped onto the balloon of a delivery device without a separate capsule that overlies and/or protects the prosthetic heart valve. One reason for this is that space is always at a premium in transcatheter prosthetic heart valve delivery devices and systems, and adding a capsule in addition to the prosthetic valve and the underlying balloon may not be feasible given the size profile requirements of these procedures.

During delivery and/or crimping, it may be possible to damage the valve assembly (e.g., the leaflet and/or cuff). Specifically, due to high forces when reducing the frame of the valve down to the desired delivery diameter, the leaflets of the valve may be pressed against the hard metal stent. This may cause localized stress and/or damage to the leaflets. Additionally, if the stent includes large open cells, the compressed leaflets may protrude through the openings between struts of the open cells and become pinched.

### BRIEF SUMMARY OF THE DISCLOSURE

In some embodiments, a prosthetic heart valve system includes a prosthetic heart valve including a stent, a cuff and a plurality of leaflets, the cuff and the plurality of leaflets forming a valve assembly, and an expandable balloon having a deflated state and an inflated state, the expandable balloon being configured and arranged to transition the prosthetic heart valve from a collapsed condition to an expanded condition and protect portions of the valve assembly during crimping and delivery.

In some embodiments, a method of delivery a prosthetic heart valve system, includes providing a prosthetic heart valve including a stent, a cuff and a plurality of leaflets, the cuff and the plurality of leaflets forming a valve assembly, and placing an expandable balloon inside the prosthetic heart valve in a deflated state, the expandable balloon having features to protect the prosthetic heart valve during crimping and delivery, and crimping the prosthetic heart valve and the expandable balloon while the features protect portions of the valve assembly.

### BRIEF DESCRIPTION OF THE DRAWINGS

FIG. 1A is a perspective view of a stent of a prosthetic heart valve according to an embodiment of the disclosure.

FIG. 1B is a schematic front view of a section of the stent of FIG. 1A.

FIG. 1C is a schematic front view of a section of a stent according to an alternate embodiment of the prosthetic heart valve of FIG. 1A.

FIGS. 1D-E are front views of the stent section of FIG. 1C in a collapsed and expanded state, respectively.

FIGS. 1F-G are side views of a portion of the stent according to the embodiment of FIG. 1C in a collapsed and expanded state, respectively.

FIG. 1H is a flattened view of the stent according to the embodiment of FIG. 1C, as if cut and rolled flat.

FIGS. 1I-J are front and side views, respectively, of a prosthetic heart valve including the stent of FIG. 1C.

FIG. 1K illustrates the view of FIG. 1H with an additional outer cuff provided on the stent.

FIGS. 2A-B illustrates a prosthetic heart valve PHV, crimped over a balloon in the deflated and inflated conditions.

FIGS. 3A-C are schematic top views of a prosthetic heart valve in use with an expandable balloon having leaflet protection features.

FIGS. 4A-B are schematic top views of another example of a prosthetic heart valve in use with an expandable balloon having leaflet protection features.

FIG. 5 is a schematic side view of a prosthetic heart valve having a protective sleeve.

FIG. 6A is a schematic top view of an expandable balloon having protrusions and pockets.

FIGS. 6B-E are schematic top views of another example of an expandable balloon, and the use of the balloon with a prosthetic heart valve.

### DETAILED DESCRIPTION

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic aortic valve, the inflow end is the end nearer the left ventricle while the outflow end is the end nearer the aorta. The intended position and orientation are used for the convenience of describing the valve disclosed herein, however, it should be noted that the use of the valve is not limited to the intended position and orientation, but may be deployed in any type of lumen or passageway. For example, although the prosthetic heart valve is described herein as a prosthetic aortic valve, the same or similar structures and features can be employed in other heart valves, such as the pulmonary valve, the mitral valve, or the tricuspid valve. Further, the term “proximal,” when used in connection with a delivery device or system, refers to a direction relatively close to the user of that device or system when being used as intended, while the term “distal” refers to a direction relatively far from the user of the device. In other words, the leading end of a delivery device or system is positioned distal to a trailing end of the delivery device or system, when being used as intended. As used herein, the terms “substantially,” “generally,” “approximately,” and “about” are intended to mean that slight deviations from absolute are included within the scope of the term so modified. As used herein, the stent may assume an “expanded state” and a “collapsed state,” which refer to the relative radial size of the stent.

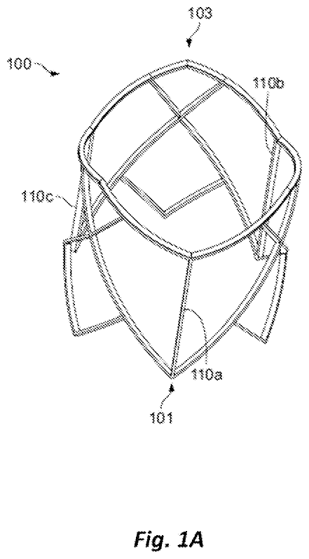

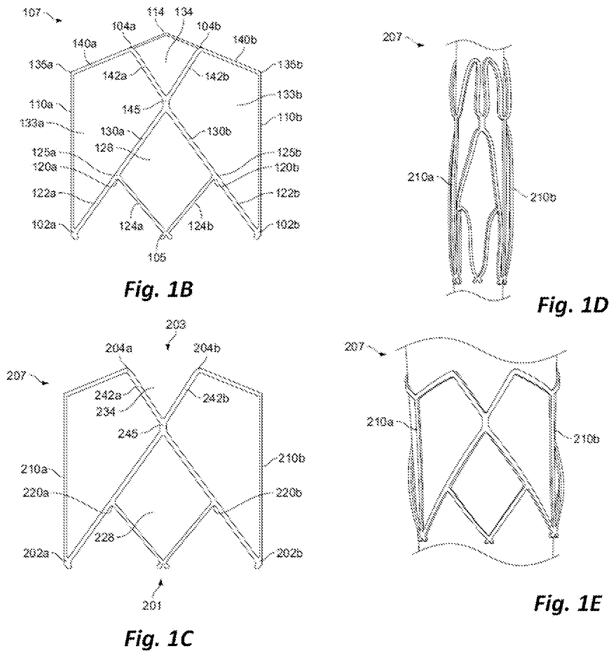

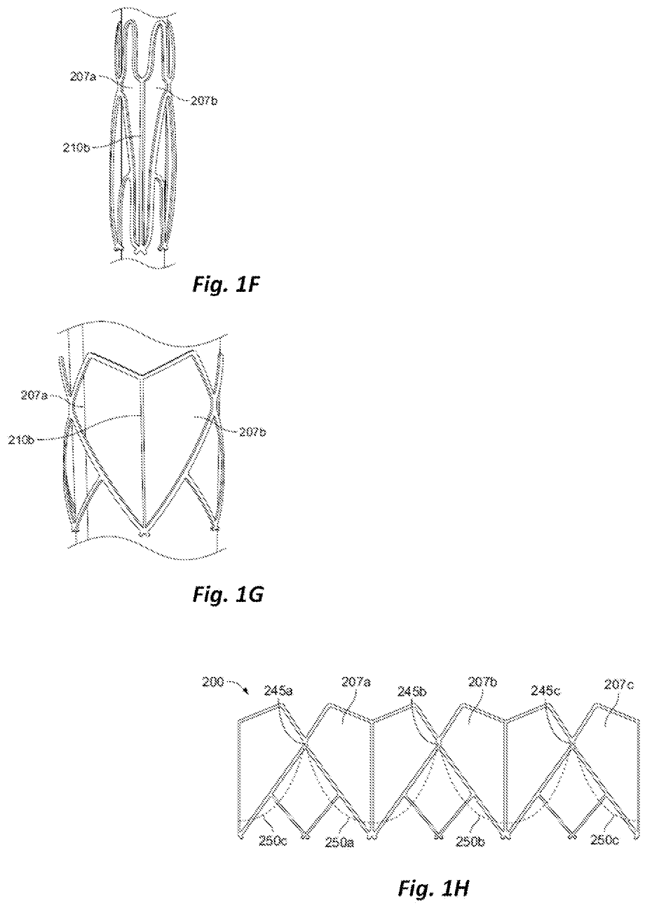

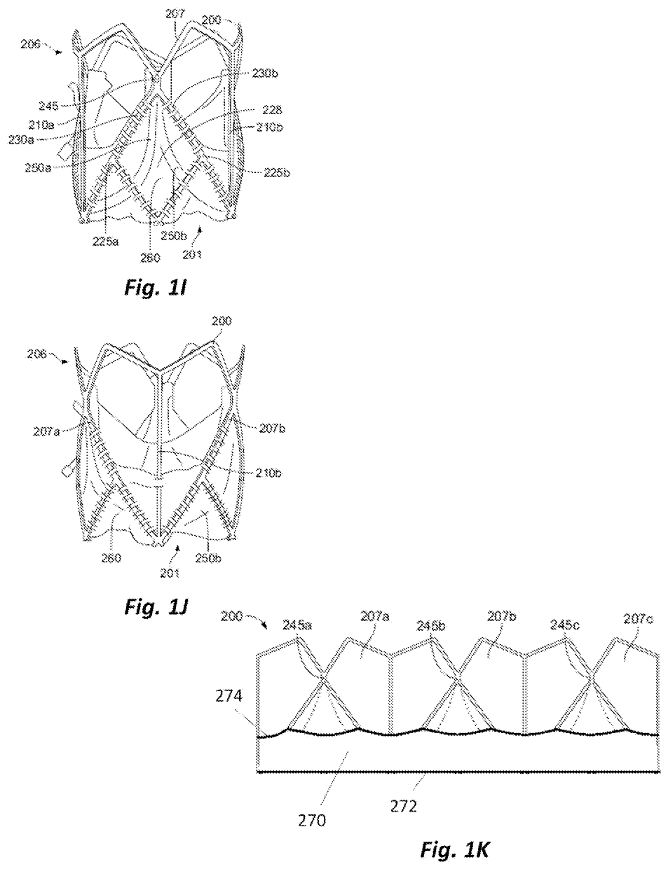

FIG. 1A illustrates a perspective view of a stent 100 of a prosthetic heart valve according to an embodiment of the disclosure. Stent 100 may include a frame extending in an axial direction between an inflow end 101 and an outflow end 103. Stent 100 includes three generally symmetric sections, wherein each section spans about 120 degrees around the circumference of stent 100. Stent 100 includes three vertical struts 110a, 110b, 110c, that extend in an axial direction substantially parallel to the direction of blood flow through the stent, which may also be referred to as a central longitudinal axis. Each vertical strut 110a, 110b, 110c may extend substantially the entire axial length between the inflow end 101 and the outflow end 103 of the stent 100, and may be disposed between and shared by two sections. In other words, each section is defined by the portion of stent 100 between two vertical struts. Thus, each vertical strut 110a, 110b, 110c is also separated by about 120 degrees around the circumference of stent 100. It should be understood that, if stent 100 is used in a prosthetic heart valve having three leaflets, the stent may include three sections as illustrated. However, in other embodiments, if the prosthetic heart valve has two leaflets, the stent may only include two of the sections.

FIG. 1B illustrates a schematic view of a stent section 107 of stent 100, which will be described herein in greater detail and which is representative of all three sections. Stent section 107 depicted in FIG. 1B includes a first vertical strut 110a and a second vertical strut 110b. First vertical strut 110a extends axially between a first inflow node 102a and a first outer node 135a. Second vertical strut 110b extends axially between a second inflow node 102b and a second outer node 135b. As is illustrated, the vertical struts 110a, 110b may extend almost the entire axial length of stent 100. In some embodiments, stent 100 may be formed as an integral unit, for example by laser cutting the stent from a tube. The term “node” may refer to where two or more struts of the stent 100 meet one another. A pair of sequential inverted V's extends between inflow nodes 102a, 102b, which includes a first inflow inverted V 120a and a second inflow inverted V 120b coupled to each other at an inflow node 105. First inflow inverted V 120a comprises a first outer lower strut 122a extending between first inflow node 102a and a first central node 125a. First inflow inverted V 120a further comprises a first inner lower strut 124a extending between first central node 125a and inflow node 105. A second inflow inverted V 120b comprises a second inner lower strut 124b extending between inflow node 105 and a second central node 125b. Second inflow inverted V 120b further comprises a second outer lower strut 122b extending between second central node 125b and second inflow node 102b. Although described as inverted V's, these structures may also be described as half-cells, each half cell being a half-diamond cell with the open portion of the half-cell at the inflow end 101 of the stent 100.

Stent section 107 further includes a first central strut 130a extending between first central node 125a and an upper node 145. Stent section 107 also includes a second central strut 130b extending between second central node 125b and upper node 145. First central strut 130a, second central strut 130b, first inner lower strut 124a and second inner lower strut 124b form a diamond cell 128. Stent section 107 includes a first outer upper strut 140a extending between first outer node 135 and a first outflow node 104a. Stent section 107 further includes a second outer upper strut 140b extending between second outer node 135b and a second outflow node 104b. Stent section 107 includes a first inner upper strut 142a extending between first outflow node 104a and upper node 145. Stent section 107 further includes a second inner upper strut 142b extending between upper node 145 and second outflow node 104b. Stent section 107 includes an outflow inverted V 114 which extends between first and second outflow nodes 104a, 104b. First vertical strut 110a, first outer upper strut 140a, first inner upper strut 142a, first central strut 130a and first outer lower strut 122a form a first generally kite-shaped cell 133a. Second vertical strut 110b, second outer upper strut 140b, second inner upper strut 142b, second central strut 130b and second outer lower strut 122b form a second generally kite-shaped cell 133b. First and second kite-shaped cells 133a, 133b are symmetric and opposite each other on stent section 107. Although the term “kite-shaped,” is used above, it should be understood that such a shape is not limited to the exact geometric definition of kite-shaped. Outflow inverted V 114, first inner upper strut 142a and second inner upper strut 142b form upper cell 134. Upper cell 134 is generally kite-shaped and axially aligned with diamond cell 128 on stent section 107. It should be understood that, although designated as separate struts, the various struts described herein may be part of a single unitary structure as noted above. However, in other embodiments, stent 100 need not be formed as an integral structure and thus the struts may be different structures (or parts of different structures) that are coupled together.

FIG. 1C illustrates a schematic view of a stent section 207 according to an alternate embodiment of the disclosure. Unless otherwise stated, like reference numerals refer to like elements of above-described stent 100 but within the 200-series of numbers. Stent section 207 is substantially similar to stent section 107, including inflow nodes 202a, 202b, vertical struts 210a, 210b, first and second inflow inverted V's 220a, 220b and outflow nodes 204a, 204b. The structure of stent section 207 departs from that of stent section 107 in that it does not include an outflow inverted V. The purpose of an embodiment having such structure of stent section 207 shown in FIG. 1C is to reduce the required force to expand the outflow end 203 of the stent 200, compared to stent 100, to promote uniform expansion relative to the inflow end 201. Outflow nodes 204a, 204b are connected by a properly oriented V formed by first inner upper strut 242a, upper node 245 and second inner upper strut 242b. In other words, struts 242a, 242b may form a half diamond cell 234, with the open end of the half-cell oriented toward the outflow end 203. Half diamond cell 234 is axially aligned with diamond cell 228. Adding an outflow inverted V coupled between outflow nodes 204a, 204b contributes additional material that increases resistance to modifying the stent shape and requires additional force to expand the stent. The exclusion of material from outflow end 203 decreases resistance to expansion on outflow end 203, which may promote uniform expansion of inflow end 201 and outflow end 203. In other words, the inflow end 201 of stent 200 does not include continuous circumferential structure, but rather has mostly or entirely open half-cells with the open portion of the half-cells oriented toward the inflow end 201, whereas most of the outflow end 203 includes substantially continuous circumferential structure, via struts that correspond with struts 140a, 140b. All else being equal, a substantially continuous circumferential structure may require more force to expand compared to a similar but open structure. Thus, the inflow end 101 of stent 100 may require more force to radially expand compared to the outflow end 103. By omitting inverted V 114, resulting in stent 200, the force required to expand the outflow end 203 of stent 200 may be reduced to an amount closer to the inflow end 201.

FIG. 1D shows a front view of stent section 207 in a collapsed state and FIG. 1E shows a front view of stent section 207 in an expanded state. It should be understood that stent 200 in FIGS. 1D-E is illustrated with an opaque tube extending through the interior of the stent, purely for the purpose of helping illustrate the stent, and which may represent a balloon over which the stent section 207 is crimped. As described above, a stent comprises three symmetric sections, each section spanning about 120 degrees around the circumference of the stent. Stent section 207 illustrated in FIGS. 1D-E is defined by the region between vertical struts 210a, 210b. Stent section 207 is representative of all three sections of the stent. Stent section 207 has an arcuate structure such that when three sections are connected, they form one complete cylindrical shape. FIGS. 1F-G illustrate a portion of the stent from a side view. In other words, the view of stent 200 in FIGS. 1F-G is rotated about 60 degrees compared to the view of FIGS. 1D-E. The view of the stent depicted in FIGS. 1F-G is centered on vertical strut 210b showing approximately half of each of two adjacent stent sections 207a, 207b on each side of vertical strut 210b. Sections 207a, 207b surrounding vertical strut 210b are mirror images of each other. FIG. 1F shows stent sections 207a, 207b in a collapsed state whereas FIG. 1G shows stent sections 207a, 207b in an expanded state.

FIG. 1H illustrates a flattened view of stent 200 including three stent sections 207a, 207b, 207c, as if the stent has been cut longitudinally and laid flat on a table. As depicted, sections 207a, 207b, 207c are symmetric to each other and adjacent sections share a common vertical strut. As described above, stent 200 is shown in a flattened view, but each section 207a, 207b, 207c has an arcuate shape spanning 120 degrees to form a full cylinder. Further depicted in FIG. 1H are leaflets 250a, 250b, 250c coupled to stent 200. However, it should be understood that only the connection of leaflets 250a-c is illustrated in FIG. 1H. In other words, each leaflet 250a-c would typically include a free edge, with the free edges acting to coapt with one another to prevent retrograde flow of blood through the stent 200, and the free edges moving radially outward toward the interior surface of the stent to allow antegrade flow of blood through the stent. Those free edges are not illustrated in FIG. 1H. Rather, the attached edges of the leaflets 250a-c are illustrated in dashed lines in FIG. 1H. Although the attachment may be via any suitable modality, the attached edges may be preferably sutured to the stent 200 and/or to an intervening cuff or skirt between the stent and the leaflets 250a-c. Each of the three leaflets 250a, 250b, 250c, extends about 120 degrees around stent 200 from end to end and each leaflet includes a belly that may extend toward the radial center of stent 200 when the leaflets are coapted together. Each leaflet extends between the upper nodes of adjacent sections. First leaflet 250a extends from first upper node 245a of first stent section 207a to second upper node 245b of second stent section 207b. Second leaflet 250b extends from second upper node 245b to third upper node 245c of third stent section 207c. Third leaflet 250c extends from third upper node 245c to first upper node 245a. As such, each upper node includes a first end of a first leaflet and a second end of a second leaflet coupled thereto. In the illustrated embodiment, each end of each leaflet is coupled to its respective node by suture. However, any coupling means may be used to attach the leaflets to the stent. It is further contemplated that the stent may include any number of sections and/or leaflets. For example, the stent may include two sections, wherein each section extends 180 degrees around the circumference of the stent. Further, the stent may include two leaflets to mimic a bicuspid valve. Further, it should be noted that each leaflet may include tabs or other structures (not illustrated) at the junction between the free edges and attached edges of the leaflets, and each tab of each leaflet may be coupled to a tab of an adjacent leaflet to form commissures. In the illustrated embodiment, the leaflet commissures are illustrated attached to nodes where struts intersect. However, in other embodiments, the stent 200 may include commissure attachment features built into the stent to facilitate such attachment. For example, commissures attachment features may be formed into the stent 200 at nodes 245a-c, with the commissure attachment features including one or more apertures to facilitate suturing the leaflet commissures to the stent. Further, leaflets 250a-c may be formed of a biological material, such as animal pericardium, or may otherwise be formed of synthetic materials, such as ultra-high molecular weight polyethylene (UHMWPE).

FIGS. 1I-J illustrate prosthetic heart valve 206, which includes stent 200, a cuff 260 coupled to stent 200 (for example via sutures) and leaflets 250a, 250b, 250c attached to stent 200 and/or cuff 260 (for example via sutures). Prosthetic heart valve 206 is intended for use in replacing an aortic valve, although the same or similar structures may be used in a prosthetic valve for replacing other heart valves. Cuff 260 is disposed on a luminal or interior surface of stent 200, although the cuff could be disposed alternately or additionally on an abluminal or exterior surface of the stent. The cuff 260 may include an inflow end disposed substantially along inflow end 201 of stent 200. FIG. 1I shows a front view of valve 206 showing one stent portion 207 between vertical struts 210a, 210b including cuff 260 and an outline of two leaflets 250a, 250b sutured to cuff 260. Different methods of suturing leaflets to the cuff as well as the leaflets and/or cuff to the stent may be used, many of which are described in U.S. Pat. No. 9,326,856 which is hereby incorporated by reference. In the illustrated embodiment, the upper (or outflow) edge of cuff 260 is sutured to first central node 225a, upper node 245 and second central node 225b, extending along first central strut 230a and second central strut 230b. The upper (or outflow) edge of cuff 260 continues extending approximately between the second central node of one section and the first central node of an adjacent section. Cuff 260 extends between upper node 245 and inflow end 201. Thus, cuff 260 covers the cells of stent portion 207 formed by the struts between upper node 245 and inflow end 201, including diamond cell 228. FIG. 1J illustrates a side view of stent 200 including cuff 260 and an outline of leaflet 250b. In other words, the view of valve 206 in FIG. 1J is rotated about 60 degrees compared to the view of FIG. 1I. The view depicted in FIG. 1J is centered on vertical strut 210b showing approximately half of each of two adjacent stent sections 207a, 207b on each side of vertical strut 210b. Sections 207a, 207b surrounding vertical strut 210b are mirror images of each other. As described above, the cuff may be disposed on the stent's interior or luminal surface, its exterior or abluminal surface, and/or on both surfaces. A cuff ensures that blood does not just flow around the valve leaflets if the valve or valve assembly are not optimally seated in a valve annulus. A cuff, or a portion of a cuff disposed on the exterior of the stent, can help retard leakage around the outside of the valve (the latter known as paravalvular leakage or “PV” leakage). In the embodiment illustrated in FIGS. 1I-J, the cuff 260 only covers about half of the stent 200, leaving about half of the stent uncovered by the cuff. With this configuration, less cuff material is required compared to a cuff that covers more or all of the stent 200. Less cuff material may allow for the prosthetic heart valve 206 to crimp down to a smaller profile when collapsed. It is contemplated that the cuff may cover any amount of surface area of the cylinder formed by the stent. For example, the upper edge of the cuff may extend straight around the circumference of any cross section of the cylinder formed by the stent. Cuff 260 may be formed of any suitable material, including a biological material such as animal pericardium, or a synthetic material such as UHMWPE.

As noted above, FIGS. 1I-J illustrate a cuff 260 positioned on an interior of the stent 200. An example of an additional outer cuff 270 is illustrated in FIG. 1K. It should be understood that outer cuff 270 may take other shapes than that shown in FIG. 1K. The outer cuff 270 shown in FIG. 1K may be included without an inner cuff 260, but preferably is provided in addition to an inner cuff 260. The outer cuff 270 may be formed integrally with the inner cuff 260 and folded over (e.g., wrapped around) the inflow edge of the stent, or may be provided as a member that is separate from inner cuff 260. Outer cuff 270 may be formed of any of the materials described herein in connection with inner cuff 260. In the illustrated embodiment, outer cuff 270 includes an inflow edge 272 and an outflow edge 274. If the inner cuff 260 and outer cuff 270 are formed separately, the inflow edge 272 may be coupled to an inflow end of the stent 200 and/or an inflow edge of the inner cuff 260, for example via suturing, ultrasonic welding, or any other suitable attachment modality. The coupling between the inflow edge 272 of the outer cuff 270 and the stent 200 and/or inner cuff 260 preferably results in a seal between the inner cuff 260 and outer cuff 270 at the inflow end of the prosthetic heart valve so that any retrograde blood that flows into the space between the inner cuff 260 and outer cuff 270 is unable to pass beyond the inflow edges of the inner cuff 260 and outer cuff 270. The outflow edge 274 may be coupled at selected locations around the circumference of the stent 200 to struts of the stent 200 and/or to the inner cuff 260, for example via sutures. With this configuration, an opening may be formed between the inner cuff 260 and outer cuff 270 circumferentially between adjacent connection points, so that retrograde blood flow will tend to flow into the space between the inner cuff 260 and outer cuff 270 via the openings, without being able to continue passing beyond the inflow edges of the cuffs. As blood flows into the space between the inner cuff 260 and outer cuff 270, the outer cuff 270 may billow outwardly, creating even better sealing between the outer cuff 270 and the native valve annulus against which the outer cuff 270 presses. The outer cuff 270 may be provided as a continuous cylindrical member, or a strip that is wrapped around the outer circumference of the stent 200, with side edges, which may be parallel or non-parallel to a center longitudinal axis of the prosthetic heart valve, attached to each other so that the outer cuff 270 wraps around the entire circumference of the stent 200.

The stent may be formed from biocompatible materials, including metals and metal alloys such as cobalt chrome (or cobalt chromium) or stainless steel, although in some embodiments the stent may be formed of a shape memory material such as nitinol or the like. The stent is thus configured to collapse upon being crimped to a smaller diameter and/or expand upon being forced open, for example via a balloon within the stent expanding, and the stent will substantially maintain the shape to which it is modified when at rest. The stent may be crimped to collapse in a radial direction and lengthen (to some degree) in the axial direction, reducing its profile at any given cross-section. The stent may also be expanded in the radial direction and foreshortened (to some degree) in the axial direction.

The prosthetic heart valve may be delivered via any suitable transvascular route, for example including transapically or transfemorally. Generally, transapical delivery utilizes a relatively stiff catheter that pierces the apex of the left ventricle through the chest of the patient, inflicting a relatively higher degree of trauma compared to transfemoral delivery. In a transfemoral delivery, a delivery device housing the valve is inserted through the femoral artery and threaded against the flow of blood to the left ventricle. In either method of delivery, the valve may first be collapsed over an expandable balloon while the expandable balloon is deflated. The balloon may be coupled to or disposed within a delivery system, which may transport the valve through the body and heart to reach the aortic valve, with the valve being disposed over the balloon (and, in some circumstance, under an overlying sheath). Upon arrival at or adjacent the aortic valve, a surgeon or operator of the delivery system may align the prosthetic valve as desired within the native valve annulus while the prosthetic valve is collapsed over the balloon. When the desired alignment is achieved, the overlying sheath, if included, may be withdrawn (or advanced) to uncover the prosthetic valve, and the balloon may then be expanded causing the prosthetic valve to expand in the radial direction, with at least a portion of the prosthetic valve foreshortening in the axial direction.

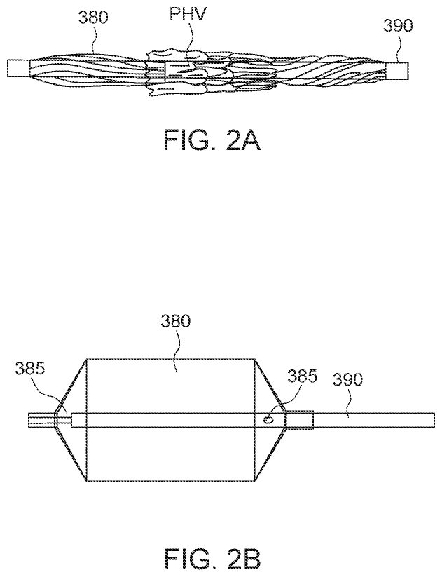

Referring to FIG. 2A, an example of a prosthetic heart valve PHV, which may include a stent similar to stents 100 or 200, is shown crimped over a balloon 380 of a balloon catheter 390 while the balloon 380 is in a deflated condition. It should be understood that other components of the delivery device, such as a handle used for steering and/or deployment, as well as a syringe for inflating the balloon 380, are omitted from FIGS. 2A-B. The prosthetic heart valve PHV may be delivered intravascularly, for example through the femoral artery, around the aortic arch, and into the native aortic valve annulus, while in the crimped condition shown in FIG. 2A. Once the desired position is obtained, fluid may be pushed through the balloon catheter 390 to inflate the balloon 380, as shown in FIG. 2B. FIG. 2B omits the prosthetic heart valve PHV, but it should be understood that, as the balloon 380 inflates, it forces the prosthetic heart valve PHV to expand into the native aortic valve annulus (although it should be understood that other heart valves may be replaced using the concepts described herein). In the illustrated example, fluid flows from a syringe or an inflation device (not shown) into the balloon 380 through a lumen within balloon catheter 390 and into one or more ports 385 located internal to the balloon 380. In the particular illustrated example of FIG. 2B, a first port 385 may be one or more apertures in a side wall of the balloon catheter 390, and a second port 385 may be the distal open end of the balloon catheter 390, which may terminate within the interior space of the balloon 380.

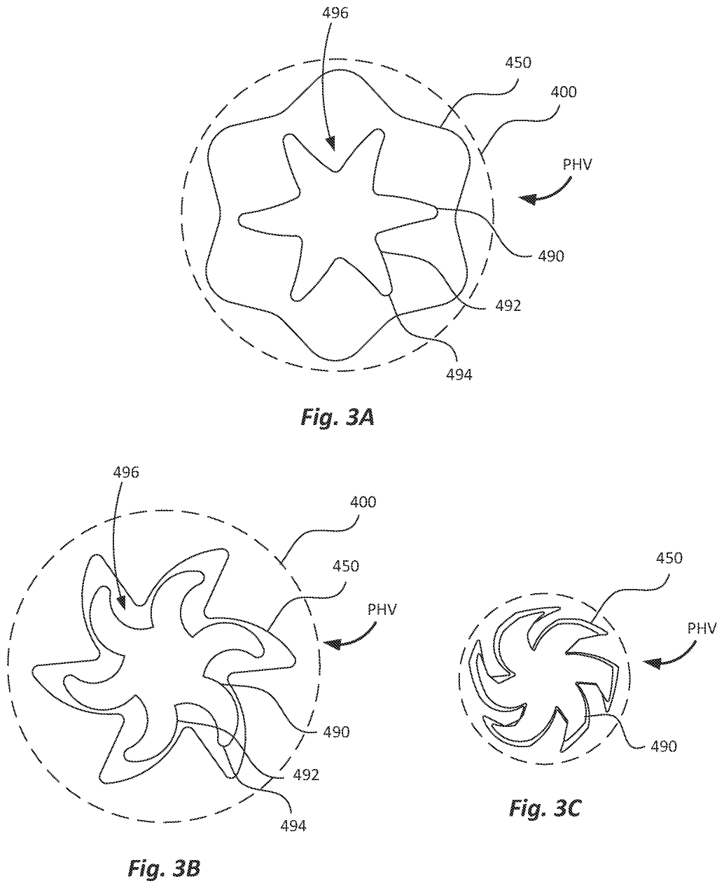

FIG. 3A illustrates one example of a prosthetic heart valve PHV having a stent 400 and a plurality of leaflets 450 that, with the cuff, form a valve assembly. For the sake of clarity, the skirt or cuff of the valve is not shown as well as the sutures that couple the leaflets and the skirt to the stent. In this configuration, an expandable balloon 490 is shown disposed within the interior of prosthetic heart valve PHV. The expandable balloon 490 may have a deflated state as shown in FIG. 3A and a generally cylindrical inflated state. Expandable balloon 490 may have an interior configured to receive a fluid (e.g., a liquid such as saline, or air) to inflate the balloon, which in turn provide sufficient radial forces to expand prosthetic heart valve PHV. Suitable materials for balloon 490 include PEBAX® elastomers, Nylon, Polyester, PET, or multilayers of various durometers of the materials listed. In the example shown, expandable balloon 490 includes a number of pleats 492 that allow the balloon to take a generally star-shaped configuration when deflated. Specifically, pleats 492 form a number of radially extending or winding arms 494, and a plurality of pockets 496 disposed between adjacent arms to receive portions of the valve assembly (e.g., portions of one or more of the leaflets) to protect the valve assembly during crimping and delivery. Pleats 492 may include V-shaped folds and in one example, three pleats 492 are provided in the expandable balloon to create three arms 494. Alternatively, six pleats 492 are provided in the expandable balloon to create six arms 494. In at least some examples, the number of pleats is equal to the number of leaflets of the valve assembly, or is correlated to the number of leaflets (e.g., the number of pleats is a multiple of the number of leaflets).

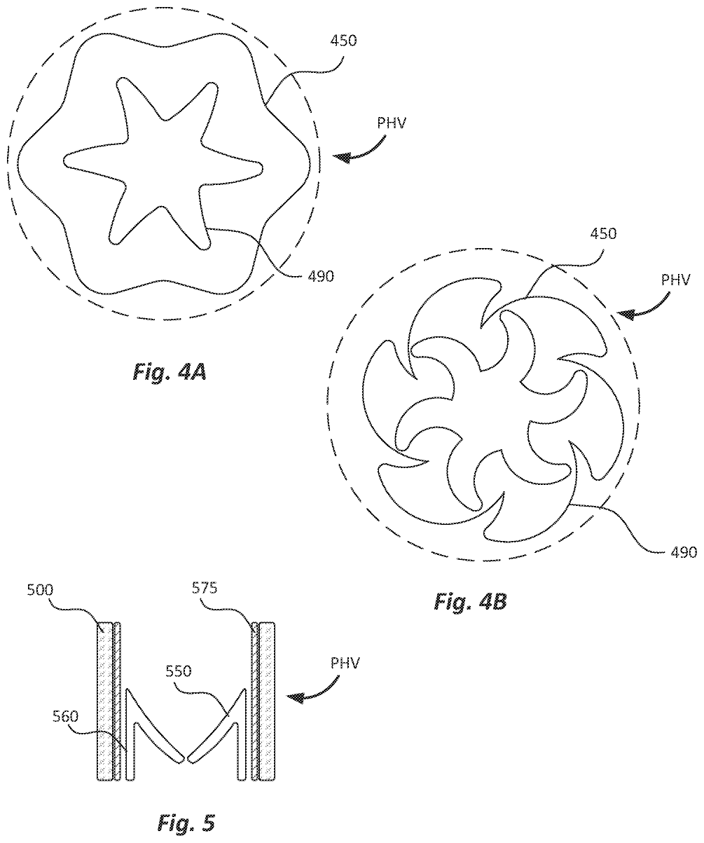

As noted, the plurality of pockets 496 are sized, configured and arranged to receive portions of the plurality of leaflets, and the arms 494 may be configured and arranged to gather and wind portions of the plurality of leaflets 450. In FIG. 3B, the star-shaped balloon 490 is generally wound in a first direction (e.g., clockwise), and the plurality of leaflets 450 is arranged to match this pattern so that they too are wound in the same direction (e.g., clockwise). It will be understood that both balloon 490 and leaflets 450 may instead be wound in a counter-clockwise direction. This spiraling or wound pattern of the leaflets and/or balloon may increase or become tighter as the prosthetic heart valve PHV is crimped to a smaller radial size for delivery (FIG. 3C). As shown in FIGS. 4A-B, it will be understood that a prosthetic heart valve PHV may also include a star-shaped balloon 490 that is wound in a first direction (e.g., counter-clockwise) and leaflets 450 that are wound in an opposite direction (e.g., clockwise).

In this manner, the expandable balloon 490 itself may double in function to both expand the prosthetic heart valve PHV and provide leaflet protection during crimping and delivery through its leaflet-protecting features (e.g., pleats and pockets). Rotation of the star-shaped or iris-shaped balloon 490 may gather portions of the leaflets during crimping and prevent or reduce the possibility of damage to the leaflet or valve assembly during crimping and delivery. With leaflets 450 gathered or entrapped between the pleats, the balloon 490 acts as a protective barrier between portions of the leaflets 450 and the interior of the stent 400. This gathering process may include gradually twisting the balloon 490 with respect to the stent, while radially crimping the prosthetic heart valve PHV over the balloon.

In addition to, or instead of the protective features of the balloon described above, a removable protective sleeve 575 may be disposed between portions of stent 500 and leaflets 550 and/or cuff 560 (FIG. 5). Removable protective sleeve 575 may be placed or introduced between the valve assembly and the stent during the crimping process and may be introduced into the body and removed during delivery along with the balloon. In at least some examples, protective sleeve 575 may be formed of PEBX® elastomers, nylon, polyethylene (HDPE, or MWPE).

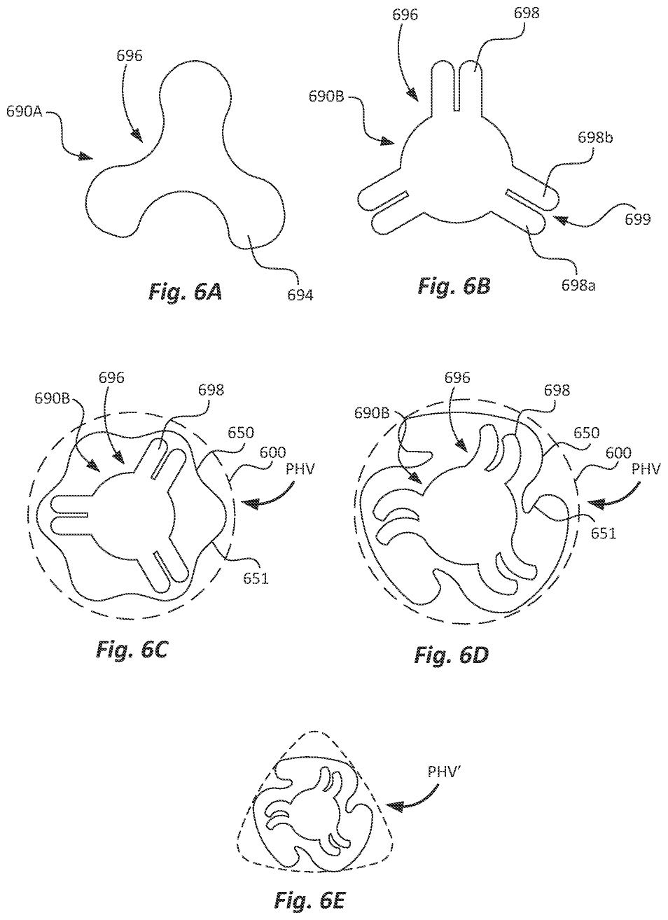

In an alternative embodiment, shown in FIG. 6A, balloon 690A may include an asymmetric configuration with a number of protrusions 694, adjacent protrusions defining interior pockets 696 sized to receive portions of the valve assembly (e.g., portions of the one or more of the leaflets) to protect the valve assembly during crimping and delivery. The balloon may be non-circular in shape to create an area that offset the peak area of the leaflets of the valve. Another example is shown in FIG. 6B, where balloon 690B includes pairs of radially extending fingers 698 that alternate with pockets 696. Specifically, each of the pairs of radially extending fingers includes a first finger 698a, a second finger 698b and a small gap 699 formed therebetween. Three pairs of fingers 698 are shown, each of the pairs being approximately 120 degrees apart from others. It will be understood that fingers 698 may be disposed alone or in pairs, as shown. Additionally, the number of fingers 698 may correspond to the number of leaflets of a prosthetic heart valve PHV. For example, three fingers 698 may be used for three leaflets. Alternatively, the fingers 698 may be a set multiple of the number of leaflets (e.g., two fingers for each leaflet, three fingers for each leaflet, etc.). The spacing between fingers or pairs of fingers may be adjusted as desired to set pocket spacing for the leaflets of the PHV.

In FIG. 6C, balloon 690B is shown disposed within the interior of prosthetic heart valve PHV, and specifically within stent 600 and the plurality of leaflets 650. The fingers 698 may be disposed between folds 651 of leaflets 650 to allow for safer and more controlled crimping. In addition to preventing or reducing damage the leaflets, these configurations may allow the device to be crimped to an even smaller size. In this, as with other embodiments, the balloon may be rotated or twisted during or before crimping to wind the leaflets and fingers of the balloon. As shown in FIG. 6D, fingers 698 are beginning to wind counterclockwise and leaflets 650 are also beginning to become wound in the same direction. In at least some examples, balloon 690B is introduced into the interior of prosthetic heart valve PHV and then wound or rotated with respect to stent 600 so that the leaflets begin to gather and wind. After winding leaflets 650 and balloon 690B, the device can be crimped to a smaller circumferential diameter. In another embodiment, the winding of balloon 690B and leaflets 650 is performed simultaneously with the crimping process instead of being a part of a sequential process. That is, the balloon 690B may be rotated relative to stent 600 and this winding may occur while prosthetic heart valve PHV is being circumferentially reduced or crimped. Based on the materials used for balloon 690B and the configuration and spacing of fingers 698, the crimped prosthetic heart valve 600 may take a non-circular fully collapsed state as shown in FIG. 6D. For example, prosthetic heart valve PHV′ is shown in a generally triangular or guitar pick shape due to the size and configuration of the balloon 690B and the overall result of the winding process of both the balloon and the leaflet 650. This concept may create larger pockets within the balloon where the bulk of the leaflet material and the valve assembly may be received. The spacing and location of these pockets thus maximize and control the packing spaced during crimping. Additionally, this configuration may reduce the forces on the leaflets to lower the possibility of damage, and may result in a non-circular crimp that may be more easily loaded, transported and eventually deployed as the commissures of the leaflets are easily located.

In use, a prosthetic heart valve may be loaded and delivered according to any of the manners and configurations described above. First, a balloon-expandable prosthetic heart valve may be provided including a stent, a cuff and a plurality of leaflets, the cuff and the plurality of leaflets forming a valve assembly. In the partially or fully expanded state of prosthetic heart valve PHV, an expandable balloon may be introduced through the interior of prosthetic heart valve PHV, the balloon being in a generally deflated state. Prosthetic heart valve PHV and the balloon may collectively form a prosthetic heart valve system. The balloon may have pleats, fingers or other leaflet-protecting features as described above, and in some instances, portions of the balloon may be disposed between portions of the valve assembly or may gather portions of the valve assembly within pockets or cavities of the balloon. Optionally, the balloon may be rotated to wind it, and to gather the leaflets therewith. The wound balloon and leaflets may then be crimped with the stent to reduce the circumferential diameter of prosthetic heart valve PHV. Alternatively, the winding of the leaflets and/or the balloon may be accomplished while crimping prosthetic heart valve PHV. It will be understood that the winding of the balloon and leaflets may be in the same direction (e.g., both clockwise or both counterclockwise) or that the winding of the balloon and the leaflets may be in different directions (e.g., a first of the balloon and leaflets is clockwise, and a second of the balloon and leaflets are wound counter-clockwise).

According to one aspect of the disclosure, a prosthetic heart valve system includes a prosthetic heart valve including a stent, a cuff and a plurality of leaflets, the cuff and the plurality of leaflets forming a valve assembly, and an expandable balloon having a deflated state and an inflated state, the expandable balloon being configured and arranged to transition the prosthetic heart valve from a collapsed condition to an expanded condition and protect portions of the valve assembly during crimping and delivery.

According to another embodiment of the disclosure, a method of delivery a prosthetic heart valve system, includes providing a prosthetic heart valve including a stent, a cuff and a plurality of leaflets, the cuff and the plurality of leaflets forming a valve assembly, and placing an expandable balloon inside the prosthetic heart valve in a deflated state, the expandable balloon having features to protect the prosthetic heart valve during crimping and delivery, and crimping the prosthetic heart valve and the expandable balloon while the features protect portions of the valve assembly.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A prosthetic heart valve system, comprising:
a prosthetic heart valve including a stent, a cuff and a plurality of leaflets, the cuff and the plurality of leaflets forming a valve assembly; and
an expandable balloon having a deflated state and an inflated state, the expandable balloon being configured and arranged to transition the prosthetic heart valve from a collapsed condition to an expanded condition and protect portions of the valve assembly during crimping and delivery.

2. The prosthetic heart valve system of claim 1, wherein the expandable balloon includes a plurality of pleats.

3. The prosthetic heart valve system of claim 2, wherein the plurality of pleats includes three pleats.

4. The prosthetic heart valve system of claim 2, wherein the plurality of pleats includes six pleats.

5. The prosthetic heart valve system of claim 2, wherein the plurality of pleats corresponds to the plurality of leaflets.

6. The prosthetic heart valve system of claim 2, wherein the plurality of pleats define a number of interior pockets configured and arranged to receive portions of the plurality of leaflets.

7. The prosthetic heart valve system of claim 1, wherein the expandable balloon is star-shaped and includes a plurality of arms in the deflated state.

8. The prosthetic heart valve system of claim 7, wherein the plurality of arms is configured and arranged to gather and wind portions of the plurality of leaflets.

9. The prosthetic heart valve system of claim 8, wherein the star-shaped balloon is wound in a first direction, and the plurality of leaflets is wound in a second direction, the first direction and the second direction being similar.

10. The prosthetic heart valve system of claim 8, wherein the star-shaped balloon is wound in a first direction, and the plurality of leaflets is wound in a second direction, the first direction and the second direction being different.

11. The prosthetic heart valve system of claim 1, further comprising a removable protective sleeve disposed between the valve assembly and the stent frame during delivery.

12. The prosthetic heart valve system of claim 1, wherein the prosthetic heart valve has a non-circular collapsed state.

13. The prosthetic heart valve system of claim 12, wherein the prosthetic heart valve has a substantially triangular collapsed state.

14. A method of delivery a prosthetic heart valve system, comprising:
providing a prosthetic heart valve including a stent, a cuff and a plurality of leaflets, the cuff and the plurality of leaflets forming a valve assembly;
placing an expandable balloon inside the prosthetic heart valve in a deflated state, the expandable balloon having features to protect the prosthetic heart valve during crimping and delivery; and
crimping the prosthetic heart valve and the expandable balloon while the features protect portions of the valve assembly.

15. The method of claim 14, further comprising rotating the expandable balloon with respect to the stent of the prosthetic heart valve prior to, or during, crimping the prosthetic heart valve.

16. The method of claim 14, wherein placing an expandable balloon comprises placing an expandable balloon having a plurality of pleats inside the prosthetic heart valve.

17. The method of claim 16, wherein placing an expandable balloon comprises placing an expandable balloon having a plurality of pleats and a number of interior pockets, and further comprising placing portions of the plurality of leaflets in the interior pockets of the expandable balloon.

18. The method of claim 14, further comprising winding the expandable balloon into a spiral configuration.

19. The method of claim 14, further comprising winding the expandable balloon in a first direction, and winding the plurality of leaflets in a second direction, the first direction and the second direction being similar.

20. The method of claim 14, further comprising winding the expandable balloon in a first direction, and winding the plurality of leaflets in a second direction, the first direction and the second direction being different.
